(19) United States
(12) Patent Application Publication
(10) Pub. No.: US 2004/0199114 A1
(43) Pub. Date: Oct. 7, 2004

(54) Intravascular heat exchange catheter with tissue preservative

(75) Inventor: Wayne Arthur Noda, Mission Viejo, CA (US)

Correspondence Address: John L. Rogitz, Esq., ROGITZ & ASSOCIATES, Suite 3120, 750 "B" Street, San Diego, CA, 92101, US

(73) Assignee: Alsius Corporation, Irvine, CA (US)

(21) Appl. No.: 10/404,028

(22) Filed: Apr. 1, 2003

(51) Int. Cl. A61F 7/12

(52) U.S. Cl. 604/113

(57) ABSTRACT

A system and method for treating a patient by inducing therapeutic hypothermia in the patient using an intravascular heat exchange catheter in conjunction with infusing tissue preservative substances into the patient.

### I. FIELD OF THE INVENTION

The present invention relates generally to intravascular heat exchange catheters.

### II. BACKGROUND OF THE INVENTION

Intravascular catheters have been introduced for controlling patient temperature. Typically, a coolant such as saline is circulated through an intravascular heat exchange catheter, which is positioned in the patient's bloodstream, to cool or heat the blood as appropriate for the patient's condition. The coolant is warmed or cooled by a computer-controlled heat exchanger that is external to the patient and that is in fluid communication with the catheter.

For example, intravascular heat exchange catheters can be used to combat potentially harmful fever in patients suffering from neurological and cardiac conditions such as stroke, subarachnoid hemorrhage, intracerebral hemorrhage, cardiac arrest, and acute myocardial infarction, or to induce therapeutic hypothermia in such patients. Further, such catheters can be used to rewarm patients after, e.g., cardiac surgery or for other reasons. Intravascular catheters afford advantages over external methods of cooling and warming, including more precise temperature control and more convenience on the part of medical personnel.

The following U.S. patents, all of which are incorporated herein by reference, disclose various intravascular catheters/systems/methods: U.S. Pat. Nos. 6,419,643, 6,416,533, 6,409,747, 6,405,080, 6,393,320, 6,368,304, 6,338,727, 6,299,599, 6,290,717, 6,287,326, 6,165,207, 6,149,670, 6,146,411, 6,126,684, 6,306,161, 6,264,679, 6,231,594, 6,149,676, 6,149,673, 6,110,168, 5,989,238, 5,879,329, 5,837,003, 6,383,210, 6,379,378, 6,364,899, 6,325,818, 6,312,452, 6,261,312, 6,254,626, 6,251,130, 6,251,129, 6,245,095, 6,238,428, 6,235,048, 6,231,595, 6,224,624, 6,149,677, 6,096,068, 6,042,559.

As critically recognized by the present invention, combining therapeutic hypothermia with tissue preservative substances can provide a heightened treatment benefit.

### SUMMARY OF THE INVENTION

A system for treating a patient includes a heat exchange catheter configured for placement in the patient to induce hypothermia in the patient when coolant is circulated through the catheter. A heat exchanger supplies coolant to the catheter and receives coolant from the catheter in a closed circuit. According to the invention, a source of tissue preservative is configured for infusing tissue preservative into the patient In a non-limiting embodiment, the source communicates with the catheter to infuse preservative therethrough into the bloodstream of the patient.

When the patient is a stroke patient, the preservative may include TPA. On the other hand, when the patient is a cardiac patient, the preservative may include one or more myocardial preservatives such as monosodiumglutamate, glucose or fructose. Additionally, infusates may contain Calcium or Potassium to regulate contractility of the heart muscle. Also, bicarbonate can serve as a buffer for maintenance of proper acid-base and fluid balance. The use of isotonic solutions such as Dextrose in Lactated Ringers can be used as a delivery solution to assure proper osmotic balance to prevent edema and cell lysis upon reperfusion.

In another aspect, a method for treating a patient includes inducing hypothermia in the patient using a closed loop heat exchange catheter, and infusing at least one tissue preservative into the patient's bloodstream.

In still another aspect, a system for treating a patient includes closed circuit heat exchange means configured for positioning in the patient to exchange heat therewith. The system also includes infusion means for infusing at least one tissue preservative into the patient's bloodstream.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a perspective view of the present intravascular heat exchange catheter, schematically showing a medicament source and coolant source in an exploded relationship with the catheter; and

FIG. 2 is a flow chart of the present method.

### DETAILED DESCRIPTION OF THE PREFERRED EMBODIMENT

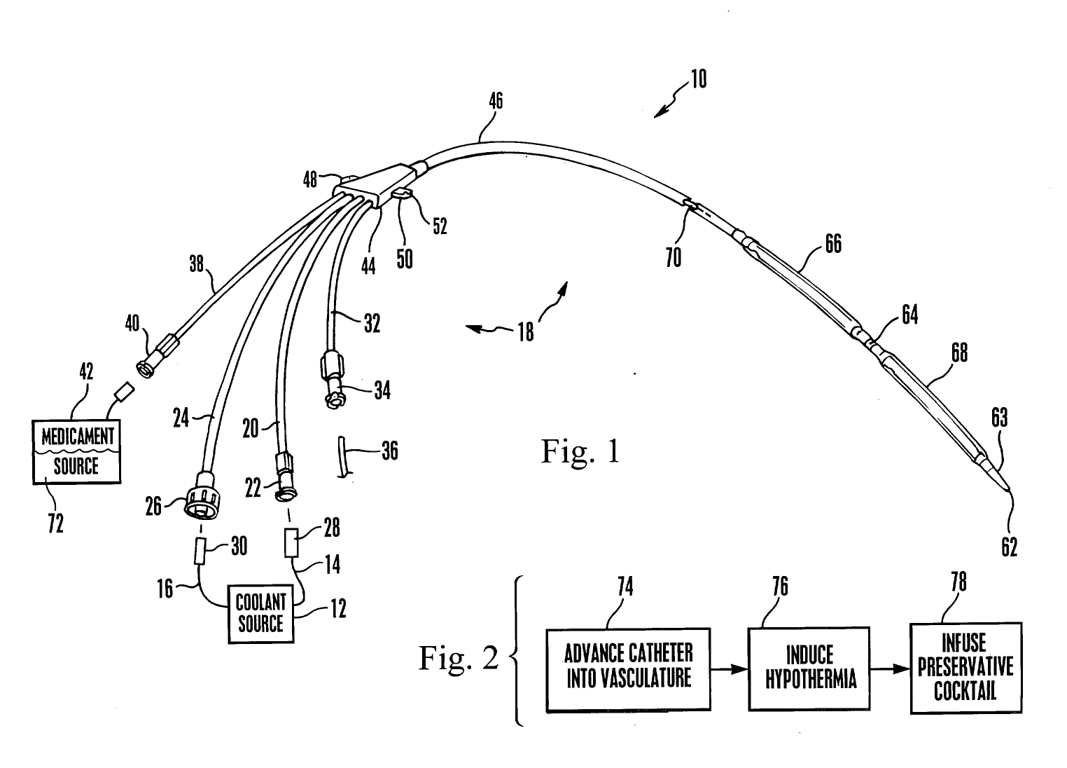

Referring initially to FIG. 1, a therapeutic catheter system, generally designated 10, is shown for establishing and maintaining hypothermia in a patient, or for attenuating a fever spike in a patient and then maintaining normal body temperature in the patient. Commencing the description of the system 10 at the proximal end, as shown the system 10 includes a coolant source 12 that can be a water-bath heat exchange system or a TEC-based heat exchange system such as any of the systems disclosed in one or more of the above-referenced patents. In any case, the coolant source provides warmed or cooled coolant such as saline through a coolant supply line 14, and coolant is returned to the source 12 via a coolant return line 16. A catheter, generally designated 18, includes a source tube 20 terminating in a fitting such as a female Luer fitting 22. Also, the catheter 18 has a return tube 24 terminating in a fitting such a male Luer fitting 26. The fittings 22, 26 can be selectively engaged with complementary fittings 28, 30 of the lines 14, 16 to establish a closed circuit coolant path between the catheter 18 and coolant source 12. The catheter 18 may be any one of the catheters set forth in the above-referenced patents. An exemplary catheter is set forth herein for illustration purposes.

Additionally, the preferred non-limiting catheter 18 includes a guide wire and primary infusion tube 32 that terminates in a fitting such as a female Luer 34. A guide wire 36 can be advanced through the tube 32 in accordance with central venous catheter placement principles, or medicament or other fluid can be infused through the guide wire and primary infusion tube 32. Moreover, a secondary infusion tube 38 with female Luer fitting 40 can be selectively engaged with a medicament source 42 for infusing fluid from the source 42 through the secondary tube 38 in accordance with present principles discussed below.

As discussed further below, the tubes 20, 24, 32, 38 are held in a distally-tapered connector manifold 44. As also set forth further below, the connector manifold 44 establishes respective pathways for fluid communication between the tubes 20, 24, 32, 38 and respective lumens in a catheter body 46.

A suture anchor 48 advantageously can be formed on the connector manifold 44 for suturing the catheter 18 to a patient. In one intended environment, the suture anchor 48 includes opposed ears 50 formed with respective suture holes 52. Other equivalent anchor structure can be used to hold the catheter 18 onto the patient, however, including surgical tape. When the catheter is a so-called Swan-Ganz catheter, i.e., a catheter of the type disclosed in U.S. Pat. No. 3,995,623, incorporated herein by reference, the anchor 48 typically would not be provided.

The exemplary catheter body 46 may include at least two lumens, and in the preferred embodiment the catheter body 46 includes at least four lumens. More specifically, the catheter body 46 can define a coolant supply lumen, a coolant return lumen, a guide wire lumen, and a secondary infusion lumen. A lumen can also be provided for holding a wire or wires that are attached to one or more distally-located sensors, such as temperature sensors, pressure sensors, gas sensors, and electrical sensors.

In any case, the connector manifold 44 establishes a pathway for fluid communication between the coolant supply tube 20 and the coolant supply lumen of the catheter. Likewise, the connector manifold 44 establishes a pathway for fluid communication between the coolant return tube 24 and the coolant return lumen. Further, the connector manifold 44 establishes a pathway for fluid communication between the guide wire and primary infusion tube 32, and the guide wire lumen, which can terminate at an open distal hole 62 defined by a distally tapered and chamfered distal tip 63 of the catheter body 46. Also, the connector manifold 44 establishes a pathway for fluid communication between the secondary infusion tube 38 and the secondary infusion lumen, which can terminate at an infusion port 64 in a distal segment of the catheter body 46. Additional ports can be provided along the length of the catheter.

The preferred non-limiting catheter 18 has a distally-located heat exchange member for effecting heat exchange with blood as it flows past the catheter when the catheter is positioned in the vasculature of a patient. The heat exchange member can be any of the heat exchange members disclosed in the above-referenced patents. By way of example, the preferred non-limiting catheter shown in FIG. 1 can have proximal and distal thin-walled heat exchange membranes 66, 68 that are arranged along the last fifteen or so centimeters of the catheter body 46 and that are bonded to the outer surface of the catheter body 46, with the infusion port 64 being located between the heat exchange membranes 66, 68. Thus, each preferred non-limiting heat exchange membrane is about six centimeters to seven and one-half centimeters in length, with the heat exchange membranes being longitudinally spaced from each other along the catheter body 46 in the preferred embodiment shown. Essentially, the heat exchange membranes 66, 68 extend along most or all of that portion of the catheter 46 that is intubated within the patient. The heat exchange membranes can be established by a medical balloon material.

The heat exchange membranes 66, 68 can be inflated with coolant from the coolant source 12 as supplied from the coolant supply lumen, and coolant from the heat exchange membranes 66, 68 is returned via the coolant return lumen to the coolant source 12. In their inflated configurations, the heat exchange membranes define a diameter of about ten French, and preferably no more than twelve French. Thus, the heat exchange membranes 66, 68 are relatively long and comparatively thin, to advantageously avoid excessively blocking blood flow through the vena cava while nevertheless effecting patient cooling.

If desired, a temperature sensor 70 such as a thermistor or other suitable device can be attached to the catheter 18 as shown. The sensor 70 can be mounted on the catheter 18 by solvent bonding at a point that is proximal to the membranes 66, 68. Or, the sensor 70 can be disposed in a lumen of the catheter 18, or attached to a wire that is disposed in a lumen of the catheter 18, with the sensor hanging outside the catheter 18. Alternatively, a separate temperature probe can be used, such as the esophageal probe disclosed in U.S. Pat. No. 6,290,717, incorporated herein by reference. As yet another alternative, a rectal probe or tympanic temperature sensor can be used. In any case, the sensor is electrically connected to the coolant source 12 for control of the temperature of the coolant as described in various of the above-referenced patents.

As envisioned by the present invention, the structure set forth above can be used in many medical applications to cool a patient and/or to maintain temperature in a normothermic or hypothermic patient, for purposes of improving the medical outcomes of patients on whom, e.g., aneurysm surgery is to be performed, preferably while the patient's temperature is below normal body temperature. The structure can then be used to rewarm the patient in a controlled manner by circulating warm coolant through the structure, or by otherwise regulating natural body rewarming by circulating coolant that is maintained at an appropriate cool (relative to normal body temperature) or warm (relative to normal body temperature) temperature through the structure.

As another example, head trauma and/or stroke can be treated by and after lowering and maintaining the patient's temperature below normal body temperature. Or, cardiac arrest can be treated while the patient's temperature is below normal body temperature. Yet again, minimally invasive heart surgery can be performed on the patient while the patient's temperature is below normal body temperature. And, myocardial infarction in the patient can be treated while the patient's temperature is below normal body temperature. Also, the present invention understands that for certain patients, e.g., stroke victims, it is important to maintain the temperature of a patient at or below normal body temperature, when the patient runs or attempts to run a fever. For severe ischemic stroke victims, the malady can be treated by maintaining the patients body temperature at a hypothermic level.

In addition to inducing therapeutic hypothermia to treat such patients, a tissue preservative agent or cocktail can be infused into the patient. If desired, the catheter 18 can be used to infuse the tissue preservative. More specifically, the medicament source 42 can hold tissue preservative 72 in liquid form, and the tissue preservative can be infused through the infusion tube 38 (or other infusion lumen) into the bloodstream of the patient while saline is circulating through the heat exchange elements 66, 68 to cool the blood without the saline entering the bloodstream.

FIG. 2 shows the present method. Commencing at block 74, the catheter 18 is advanced into the patient, preferably into the vasculature (e.g., vena cava) but potentially into a body cavity such as the bladder, rectum, or esophagus. The catheter 18 is then used at block 76 to induce hypothermia in the patient by circulating cold saline or other heat exchange fluid through the catheter 18 without allowing the saline or fluid to enter the bloodstream. The patient preferably may be cooled to mild or moderate hypothermia ranges.

At block 78, either before, during, or after hypothermia is induced, the tissue preservative is infused into the patient. As stated above, the tissue preservative may be infused through the catheter 18 or through an ancillary infusion device (not shown).

Non-limiting examples of tissue preservatives that may be used include substances such as TPA for preserving brain tissue and substances for preserving heart tissue. Examples of heart tissue preservatives include crystalloid solutions containing substances such as monosodiumglutamate, glucose, etc. that can be infused into the bloodstream ideally through the catheter to optimize delivery nearby the heart thus minimize the dilution to the rest of the body. If the catheter 18 is placed partially in the heart, the preservative can be infused directly into the heart. In any case, it will be appreciated that by “preservative” is meant substances that can replenish energy to the heart muscle cells, substances such as buffers that prevent cells from exploding upon, e.g., reperfusion, and substances that otherwise stimulate heart tissue or revitalize the tissue. Sugars such as glucose and fructose belong in the latter group.

While the particular INTRAVASCULAR HEAT EXCHANGE CATHETER WITH TISSUE PRESERVATIVE as herein shown and described in detail is fully capable of attaining the above-described objects of the invention, it is to be understood that it is the presently preferred embodiment of the present invention and is thus representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more”. All structural and functional equivalents to the elements of the above-described preferred embodiment that are known or later come to be known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. §112, sixth paragraph, unless the element is expressly recited using the phrase “means for”.

What is claimed is:

1. A system for treating a patient, comprising:
a heat exchange catheter configured for placement in the patient to induce hypothermia in the patient when coolant is circulated through the catheter;
a heat exchanger supplying coolant to the catheter and receiving coolant from the catheter in a closed circuit; and
at least one source of tissue preservative configured for infusing tissue preservative into the patient.

2. The system of claim 1, wherein the source communicates with the catheter to infuse preservative therethrough into the bloodstream of the patient.

3. The system of claim 1, wherein the patient is a stroke patient, and the preservative includes TPA.

4. The system of claim 1, wherein the patient is a cardiac patient, and the preservative includes at least one substance for preserving heart tissue.

5. The system of claim 4, wherein the substance includes at least one crystalloid.

6. The system of claim 4, wherein the substance includes at least one of:
monosodiumglutamate and a sugar.

7. The system of claim 4, wherein the substance includes at least one of: Potassium, and Calcium.

8. The system of claim 4, wherein the substance includes a buffer.

9. The substance of claim 8, wherein the substance includes bicarbonate to maintain fluid balance.

10. A method for treating a patient, comprising:
inducing hypothermia in the patient using a closed loop heat exchange catheter; and
infusing at least one tissue preservative into the patient's bloodstream.

13. The method of claim 12, wherein the act of infusing is undertaken using the catheter.

14. The method of claim 12, wherein the patient is a stroke patient and the preservative includes TPA.

15. The method of claim 12, wherein the patient is a cardiac patient, and the preservative includes at least one substance selected from the group consisting of: crystalloids, myocardial preservative substances.

16. A system for treating a patient, comprising:
closed circuit heat exchange means configured for positioning in the patient to exchange heat therewith; and
infusion means for infusing at least one tissue preservative into the patient's bloodstream.

17. The system of claim 16, wherein the closed circuit heat exchange means establishes the infusion means.

18. The system of claim 16, wherein the patient is a stroke patient and the preservative includes TPA.

19. The system of claim 16, wherein the patient is a cardiac patient, and the preservative includes at least one substance selected from the group consisting of: crystalloids, Potassium Chloride (KCl), monosodiumglutamate, buffer substances that prevent cells from exploding, and at least one sugar.
